(19) United States
(12) Patent Application Publication
(10) Pub. No.: US 2022/0401147 A1
(43) Pub. Date: Dec. 22, 2022

(54) ENERGY-DELIVERY DEVICES

(71) Applicant: Covidien LP, Mansfield, MA (US)

(72) Inventors: Shujun Tang, Shanghai (CN); Yong Feng, Shanghai (CN); Peng Hou, Shanghai (CN); Wei Zhang, Shanghai (CN); Zhi Li, Shanghai (CN); Jianfeng Gu, Shanghai (CN)

(21) Appl. No.: 17/624,281

(22) PCT Filed: Jul. 3, 2019
(86) PCT No.: PCT/CN2019/094583

§ 371 Date: Dec. 31, 2021

(51) Int. Cl. A61B 18/18

(52) U.S. Cl. CPC A61B 18/1815; A61B 2018/1869; A61B 2018/00023

(57) ABSTRACT

An energy-delivery device (10) includes a handle body (12), an antenna assembly (14) extending distally from the handle body (12), and a transmission line (16) configured to be detachably coupled to the antenna assembly (14).

### INTRODUCTION

The present technology is related generally to energy-delivery devices suitable for use in tissue ablation applications.

### BACKGROUND

Treatment of certain diseases requires the destruction of malignant tissue growths, e.g., tumors. Treatment may involve inserting ablation probes into tissues where cancerous tumors have been identified. Once the probes are positioned, electromagnetic energy is passed through the probes into surrounding tissue.

Electrosurgical devices utilizing electromagnetic radiation have been developed for a variety of uses and applications. Typically, these devices include a power generation source, e.g., a microwave or radio frequency (RF) electrosurgical generator that functions as an energy source, and a surgical instrument having a microwave ablation probe having an antenna assembly for directing energy to the target tissue. The generator and surgical instrument are typically operatively coupled by a cable assembly having a plurality of conductors for transmitting energy from the generator to the instrument, and for communicating control, feedback and identification signals between the instrument and the generator.

### SUMMARY

In one aspect, the present disclosure provides an energy-delivery device for delivering energy to tissue. The energy-delivery device includes a handle body, an antenna assembly coupled to the handle body and extending distally therefrom, a transmission line, and first and second mating parts. The transmission line has a first end portion configured to be coupled to the handle body, and a second end portion configured to be coupled to an energy source. The first mating part is coupled to the handle body or the first end portion of the transmission line, and the second mating part is coupled to the other of the handle body or the first end portion of the transmission line. The second mating part is configured to be detachably coupled to the first mating part to detachably couple the transmission line to the antenna assembly.

In aspects, the first mating part may define a recess, and the second mating part may have a projection configured for receipt in the recess to couple a coaxial cable of the transmission line to a feedline of the antenna assembly.

In aspects, the first mating part may define an aperture, and the second mating part may have a node configured to be received within the aperture to couple a thermocouple wire of the transmission line to a temperature sensor of the antenna assembly.

In aspects, the projection may include a plurality of tabs arranged in a circular array. The tabs may be configured to flex radially inward upon receipt in the recess.

In aspects, the first mating part may include a rod centrally disposed within the recess, and the second mating part may include a tubular member disposed within the plurality of tabs. The tubular member may be configured to receive the rod.

In aspects, the first mating part may define a pair of depressions disposed on opposite sides of the recess, and the second mating part may have a pair of surface features disposed on opposite sides of the projection. The pair of depressions may be configured to receive the corresponding pair of surface features.

In aspects, the first mating part may be disposed at a proximal-facing end of the handle body and face a proximal direction.

In aspects, the first mating part may be disposed at a proximal portion of the handle body and face a direction that is perpendicular to a longitudinal axis defined by the antenna assembly.

In aspects, the first and second mating parts may be magnetically attracted.

In aspects, the second mating part may be rotatable relative to the first mating part while remaining coupled to the first mating part.

In aspects, the first mating part may define a ring-shaped channel, and the second mating part may have a node configured for receipt in the ring-shaped channel.

In aspects, the first mating part may define a pair of slits, and the second mating part may have a pair of flexible arms configured for receipt in the corresponding pair of slits.

In another aspect of the disclosure, the disclosure provides a microwave-energy delivery device including a handle body, an antenna assembly coupled to the handle body and extending distally therefrom, a transmission line, and first and second mating parts. The transmission line has a first end portion configured to be coupled to the handle body, and a second end portion configured to be coupled to an energy source. The first mating part is coupled to the handle body and defines a recess, and the second mating part is coupled to the first end portion of the transmission line. The second mating part has a projection configured for receipt in the recess. The first and second mating parts are magnetically attracted to one another and configured to be detachably coupled to detachably couple the transmission line to the antenna assembly.

In yet another aspect, the disclosure provides a method of using a microwave-energy delivery device is provided. An antenna assembly of a microwave-energy delivery device is inserted into a body while the antenna assembly is detached from a transmission line of the microwave-energy delivery device. A first mating part disposed at a first end portion of the transmission line is coupled to a second mating part disposed at a handle body of the microwave-energy delivery device after the antenna assembly is inserted into the body. Coupling the first and second mating parts couples a coaxial cable of the transmission line to a feedline of the antenna assembly and a thermocouple wire of the transmission line to a temperature sensor of the antenna assembly

In aspects, coupling the first and second mating parts may include magnetically coupling the first and second mating parts.

In aspects, coupling the first and second mating parts may include inserting a projection of the first mating part into a recess defined by the second mating part.

The details of one or more aspects of the disclosure are set forth in the accompanying drawings and the description below. Other features, objects, and advantages of the techniques described in this disclosure will be apparent from the description and drawings, and from the claims.

The term “proximal” refers to that portion of the device, or component thereof, closer to the user; and the term “distal” refers to that portion of the device, or component thereof, farther from the user.

Electromagnetic energy is generally classified by increasing energy or decreasing wavelength into radio waves, microwaves, infrared, visible light, ultraviolet, X-rays and gamma-rays. As it is used in this description, “microwave” generally refers to electromagnetic waves in the frequency range of 300 megahertz (MHz) (3×108 cycles/second) to 300 gigahertz (GHz) (3×1011 cycles/second).

### BRIEF DESCRIPTION OF THE DRAWINGS

Objects and features of the presently disclosed energy-delivery devices will become apparent to those of ordinary skill in the art when descriptions of various embodiments thereof are read with reference to the accompanying drawings, of which:

FIG. 1A is a perspective view illustrating an energy-delivery device with a transmission line coupled to a proximal portion of a handle assembly;

FIG. 1B is a perspective view illustrating the energy-delivery device of FIG. 1A with the transmission line detached from the handle assembly;

FIG. 2 is a longitudinal cross-section of the energy-delivery device of FIG. 1A illustrating a mating part of the transmission line coupled to a mating part of the handle assembly;

FIG. 3A is a perspective view, with a portion of an outer handle body removed, illustrating details of the mating part of the handle assembly;

FIG. 3B is a perspective view, with a portion of the outer handle body removed, illustrating details of the mating part of the transmission line;

FIG. 4A is a perspective view illustrating another embodiment of an energy-delivery device with the transmission line decoupled from a proximal portion of the handle assembly;

FIG. 4B is a perspective view illustrating the energy-delivery device of FIG. 4A with the transmission line coupled to the handle assembly;

FIG. 5 is a perspective view illustrating another embodiment of first and second mating parts for coupling a transmission line to a handle assembly of any of the embodiments of the disclosed energy-delivery devices;

FIG. 6 is an enlarged view illustrating details of the first mating part of FIG. 5 coupled to a transition block of the handle assembly;

FIG. 7 is an exploded view illustrating the first mating part of FIG. 6;

FIG. 8 is a perspective view illustrating details of the second mating part of FIG. 5 coupled to the transmission line;

FIG. 9 is a perspective view illustrating another embodiment of first and second mating parts for coupling a transmission line and handle assembly of any of the embodiments of the disclosed energy-delivery devices;

FIG. 10 is a perspective view illustrating the first and second mating parts of FIG. 9 decoupled from one another;

FIG. 11 is a perspective view illustrating the second mating part of FIG. 9 coupled to the transmission line; and

FIG. 12 is a longitudinal cross section of the second mating part of FIG. 11.

### DETAILED DESCRIPTION

The disclosure is generally directed to microwave-energy delivery devices including a handle assembly, an antenna assembly supported by the handle assembly, and a transmission line for transmitting microwave energy. The transmission line includes a mating part configured to be detachably coupled to a corresponding mating part of the handle assembly. Since the respective mating parts of the transmission line and the handle assembly are detachably coupled, a clinician may navigate the antenna assembly into a body cavity of a patient during a surgical procedure without having the transmission line coupled to the handle assembly. Upon positioning the antenna assembly at a selected location, the transmission line may then be coupled to the handle assembly, whereupon the device may be activated to cause the coupled transmission line to transmit microwave energy to the antenna assembly for treating tissue. These and other aspects of the present disclosure are described in greater detail below.

Various embodiments of the presently disclosed energy-delivery devices are suitable for microwave or RF ablation and for use to pre-coagulate tissue for microwave or RF ablation-assisted surgical resection. Although various methods described hereinbelow are targeted toward microwave ablation and the complete destruction of target tissue, it is to be understood that methods for directing electromagnetic radiation may be used with other therapies in which the target tissue is partially destroyed or damaged.

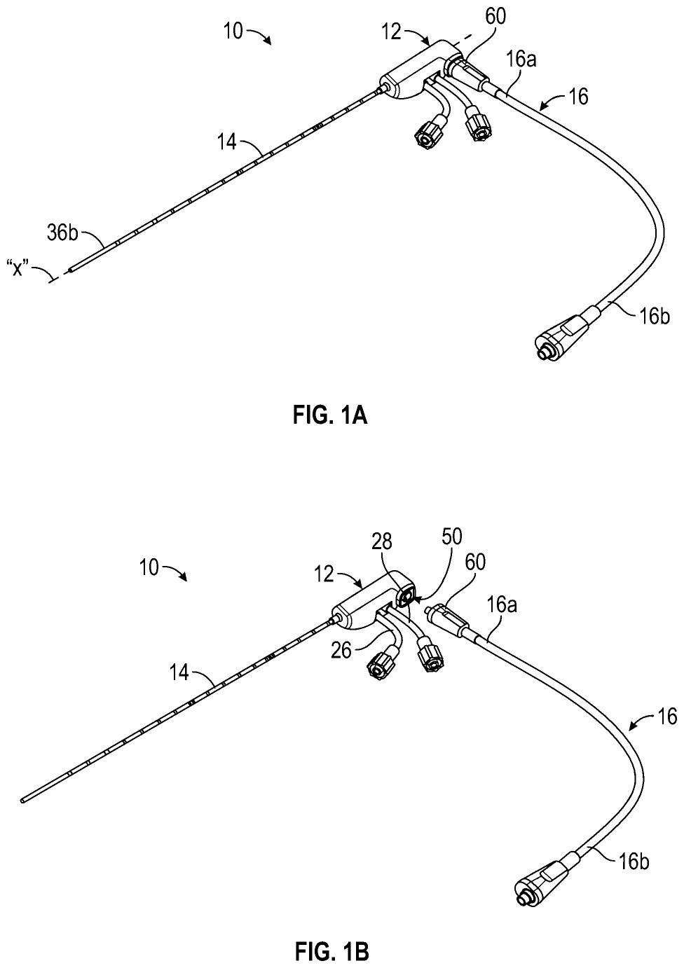

FIGS. 1A and 1B illustrate an exemplary energy-delivery device 10 shown in an assembled state and a disassembled state, respectively. The energy-delivery device 10 generally includes a handle assembly 12, an antenna assembly 14 supported by the handle assembly 12, and a transmission line 16 for transmitting electrosurgical energy (e.g., microwave energy, RF energy, etc.) to the antenna assembly 14. The energy-delivery device 10 is configurable between a fully assembled state (FIG. 1A), in which the transmission line 14 is electrically and mechanically coupled to the antenna assembly 14 either directly or via the handle assembly 12, and a disassembled state (FIG. 1B), in which the transmission line 14 is decoupled from the antenna assembly 14.

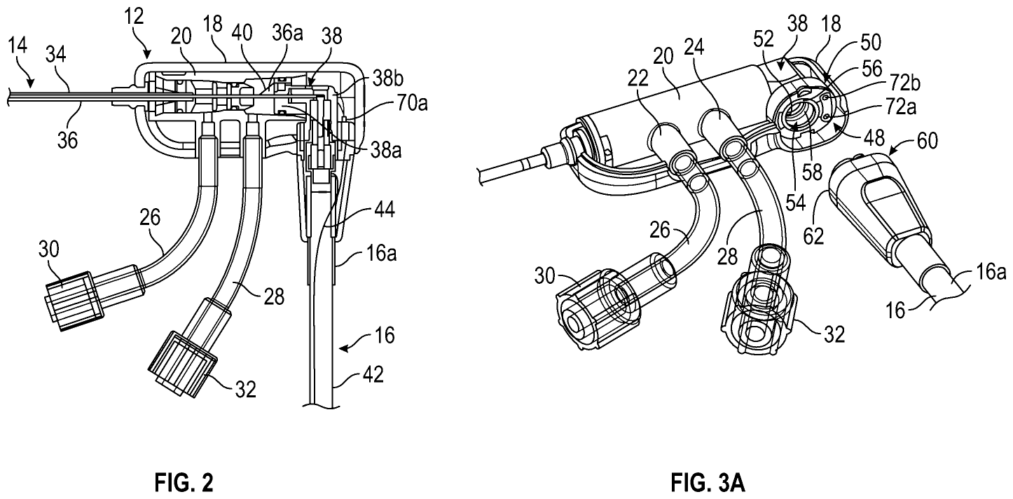

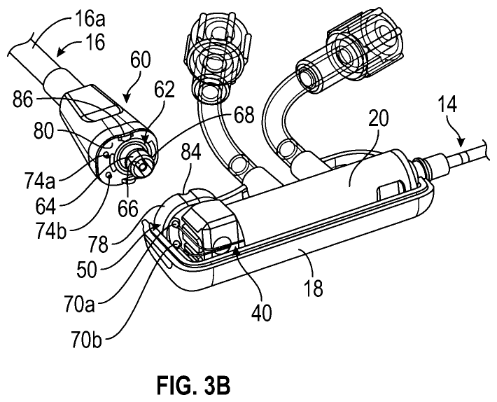

With additional reference to FIGS. 2, 3A, and 3B, the handle assembly 12 has an outer handle body 18 and an inner handle body 20 disposed within the outer handle body 18. The inner handle body 20 includes first and second ports 22, 24 extending therefrom. The first port 22 is coupleable to a supply line 26 configured to deliver cooling fluid to the antenna assembly 14 via the first port 22, and the second port 24 is coupleable to a return line 28 configured to receive the cooling fluid from the antenna assembly 14 returning via the second port 24 after the cooling fluid has circulated through the antenna assembly 14. Each of the supply and return lines 26, 28 has a respective luer connector 30, 32 at an end thereof for connection to a cooling fluid source (not shown). The cooling fluid source may include a cooling unit capable of actively cooling the cooling fluid returning from the antenna assembly 12 via the second port 24. In aspects, the ends of the supply and return lines 26, 28 may have any connector suitable for connection to a fluid source.

The antenna assembly 14 includes an outer tubular member 34 and a feedline 36 disposed therein. The outer tubular member 34 may be formed of any suitable non-electrically-conductive material, such as, for example, polymeric or ceramic materials. The feedline 36 may be a coaxial cable including an inner conductor, an outer conductor coaxially disposed around the inner conductor, and a dielectric material disposed therebetween. The antenna assembly 14 includes a transition block 38 disposed within the outer handle body 18 that serves to electrically couple the transmission line 16 to the feedline 36, thereby enabling transmission of microwave energy from the transmission line 16 to the feedline 36. With this purpose in mind, the transition block 38 includes a first end portion 38a coupled to a proximal end portion 36a of the feedline 36 and a second end portion 38b coupled to the transmission line 16, as described in more detail below. In embodiments, the first and second end portions 38a, 38b of the transition block 38 may be oriented at any suitable angle relative to one another, such as, for example, a right angle. The antenna assembly 14 further includes a temperature sensor 40 extending generally along a longitudinal axis of the feedline 36 and terminating distally at a distal end portion 36b (FIG. 1A) of the feedline 36. In some embodiments, the temperature sensor 40 may be a thermocouple.

The transmission line 16 has a first end portion 16a configured to be detachably coupled to the antenna assembly 14, and a second end portion 16b (FIGS. 1A and 1B) configured to couple the antenna assembly 14 to an electrosurgical generator (not shown), such as, for example, a microwave generator. The transmission line 16 may include a coaxial cable 42 configured to be coupled to the feedline 36 of the antenna assembly 14, and a thermocouple wire 44 configured to be electrically coupled to the temperature sensor 40 of the antenna assembly 14. The coaxial cable 42 includes an inner conductor, a dielectric material coaxially surrounding the inner conductor, and an outer conductor coaxially surrounding the dielectric material. The thermocouple wire 44 may be a two-lead thermocouple wire including an insulated (anodized) wire and a copper wire. As will be described, the transmission line 16 is configured to be detachable from the antenna assembly 14, such that the coaxial cable 42 and the thermocouple wire 44 are detachable from the feedline 16 and temperature sensor 40, respectively.

With reference to FIGS. 3A and 3B, the energy-delivery device 10 includes a first mating part 50 coupled to (e.g., formed with or connected to) the transition block 38 of the handle assembly 12, and a second mating part 60 coupled to (e.g., formed with or connected to) the first end portion 16a of the transmission line 16. In embodiments, the first mating part 50 may be coupled to any suitable portion of the outer or inner handle bodies 18, 20 or the antenna assembly 14. The first mating part 50 may be disposed within the outer handle body 18 and recessed from an access opening 48 defined in an outer surface of the outer handle body 18. The access opening 48 is configured to closely surround an outer periphery of the second mating part 60 of the transmission line 16 upon coupling of the first mating part 50 with the second mating part 60. The first mating part 50 is disposed at a proximal portion of the inner handle body 20. In some embodiments, the first mating part 50 faces a direction that is perpendicular to a longitudinal axis “X” (FIG. 1A) defined by the feedline 36.

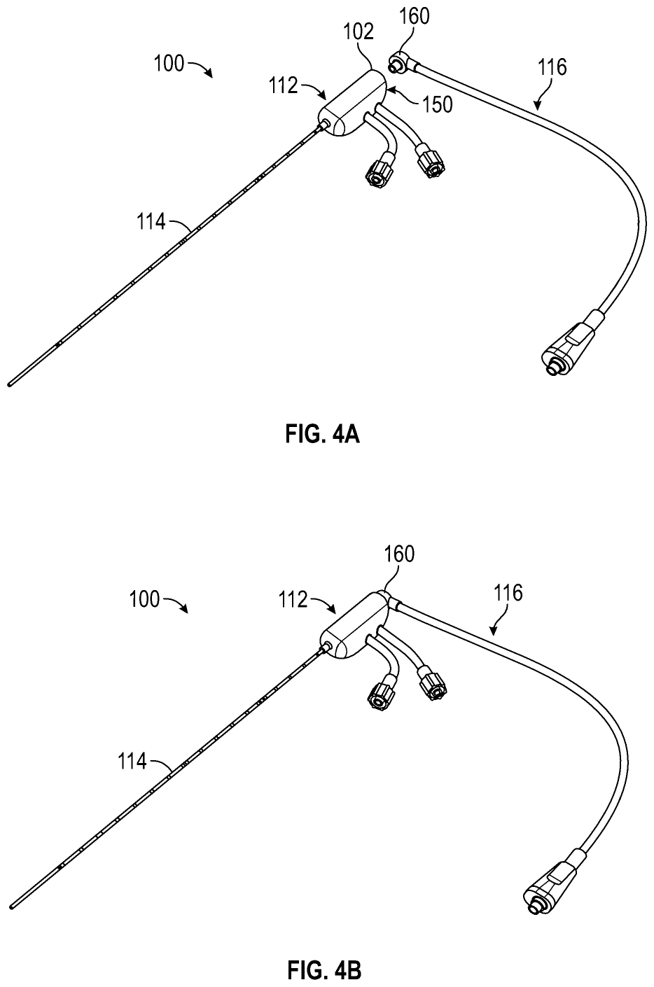

With brief reference to FIGS. 4A and 4B, another embodiment of an energy-delivery device 100, similar to the energy-delivery device 10, is illustrated and includes a first mating part 150 disposed at a proximal-facing end portion 102 of the inner handle body of the handle assembly 112 and facing a proximal direction. In this embodiment, the mating part 160 on the transmission line 116 is oriented laterally spaced from a longitudinal axis of the transmission line 116 rather than along the longitudinal axis of the transmission line 16, as is the second mating part 60 of the transmission line 16 of FIGS. 3A and 3B.

With continued reference to FIGS. 3A and 3B, the first mating part 50 includes an inner annular surface 52 defining a circular recess 54 and a rod 56 centrally disposed within the circular recess 54. In some aspects, the recess 54 may assume any suitable shape, such as, for example, a square, triangle, or the like. The inner annular surface 52 of the first mating part 50 has a circumferentially-extending ridge 58 protruding into the recess 54. The rod 56 is in electrical communication with the feedline 36 of the antenna assembly 12.

The second mating part 60 has a projection 62 extending from the first end portion 16a of the transmission line 16 and configured for receipt in the recess 54 to couple the coaxial cable 42 of the transmission line 16 to the feedline 36 of the antenna assembly 14. The projection 62 includes a plurality of tabs 64 arranged in a circular array about a tubular member 66 of the second mating part 60. The plurality of tabs 64 are configured to flex radially inward upon receipt in the recess 54 of the first mating part 50. Each of the tabs 64 has a bulging free end 68 that passes over the ridge 58 of the first mating part 50 upon receipt of the projection 62 in the recess 54, whereupon the projection 62 snap-fittingly engages the inner annular surface 52 of the first mating part 50. Concurrently with the projection 62 being received in the recess 54, the tubular member 66 of the second mating part 60 receives the rod 56 of the first mating part 50 to electrically couple the coaxial cable 42 of the transmission line 16 with the feedline 36 of the antenna assembly 14.

The inner annular surface 58 of the first mating part 50 and the projection 62 of the second mating part 60 may be fabricated from a magnetic material (e.g., iron, nickel, cobalt, alnico, ferrite etc.). In embodiments, both the first and second mating parts 50, 60 are permanent magnets, or one of the first or second mating parts 50, 60 is a permanent magnet and the other of the first or second mating parts 50, 60 is made of a ferromagnetic material. Other or all portions of the first and second mating parts 50, 60 may be made of or coated with a magnetic material, such as, for example, the receptacles 70a, 70b and nodes 74a, 74b, the rod 56 and the tubular member 66, and/or planar contact surfaces 78, 80 (FIG. 3B) of the respective first and second mating parts 50, 60. The magnetic attraction between the first and second mating parts 50, 60 assists in maintaining the transmission line 16 and the antenna assembly 14 fixed to one another while allowing for detachment thereof upon the application of a threshold separation force.

The first mating part 50 defines a pair of conductive receptacles 70a, 70b each defining an aperture 72a, 72b (FIG. 3A). The receptacles 70a, 70b are each connected to a proximal end portion of a respective wire of the temperature sensor 40. The second mating part 60 has a pair of conductive nodes 74a, 74b configured to be received within the respective pair of apertures 72a, 72b. As such, upon the pair of receptacles 70a, 70b of the first mating part 50 receiving the pair of nodes 74a, 74b of the second mating part 60, the two wires of the thermocouple wire 44 of the transmission line 16 are detachably, electrically coupled to the two wires of the temperature sensor 40 of the antenna assembly 14.

The planar contact surface 78 (FIG. 3B) of the first mating part 50 may define a pair of depressions 84 therein disposed on opposite sides of the recess 54. The planar contact surface 80 of the second mating part 60 may have a pair of protrusions 86 disposed on opposite sides of the projection 62. The pair of depressions 84 is configured to receive the corresponding pair of protrusions 86. Accordingly, the depressions 84 and protrusions 86 together function as a poka-yoke feature to ensure proper alignment of the nodes 74a, 74b of the second mating part 60 with the apertures 72a, 72b in the first mating part 50.

In operation, with the transmission line 16 decoupled from the handle assembly 12, the antenna assembly 14 is inserted into a body of a patient toward a target location (e.g., a section of liver). Due to the transmission line 16 being decoupled from the handle assembly 12, navigating the antenna assembly 14 to the target location is made easier and less cumbersome for the clinician. Upon suitable positioning of the antenna assembly 14 at the target location, the transmission line 16 may then be coupled to the handle assembly 12, thereby enabling transmission of microwave energy from the transmission line 16 to the feedline 36. More specifically, the plurality of tabs 64 of the second mating part 60 of the transmission line 16 are received in the recess 54 of the first mating part 50 and the tubular member 66 of the second mating part 60 is positioned over the rod 56 of the first mating part 50 to couple the first mating part 50 to the second mating part 60. Since the coaxial cable 42 of the transmission line 16 is connected to the tubular member 66 of the second mating part 60, and the feedline 36 of the antenna assembly 14 is connected to the rod 56 of the first mating part 50, the coaxial cable 42 of the transmission line 16 and the feedline 36 of the antenna assembly 14 are electrically coupled upon coupling the first mating part 50 with the second mating part 60. Substantially concurrently with coupling the projection 62 and tubular member 66 of the second mating part 60 with the corresponding recess 54 and rod 56 of the first mating part 50, the nodes 74a, 74b of the second mating part 60 are received in the receptacles 70a, 70b of the first mating part 50. Due to the individual wires of the thermocouple wire 44 of the transmission line 16 being connected to the nodes 74a, 74b, and the individual wires of the temperature sensor 40 of the antenna assembly 14 being connected to the receptacles 70a, 70b, the thermocouple wire 44 and the temperature sensor 40 are electrically coupled upon coupling the first mating part 50 with the second mating part 60.

With the transmission line 16 coupled to the antenna assembly 14, the device 10 may be activated to deliver electrosurgical energy to the target location to treat tissue. After treating the tissue, it may be desirable to either relocate the antenna assembly 14 (e.g., reposition the tip of the antenna assembly 14 within the surgical site) to other target locations to treat tissue or to remove the antenna assembly 14 altogether from the surgical site. To make it easier for the clinician to move the antenna assembly 14, the transmission line 16 may be decoupled from the antenna assembly 14 by simply overcoming the magnetic attraction between the first and second mating parts 50, 60 and the holding force of the inner annular surface 58 of the first mating part 50 on the tabs 64 of the second mating part 60.

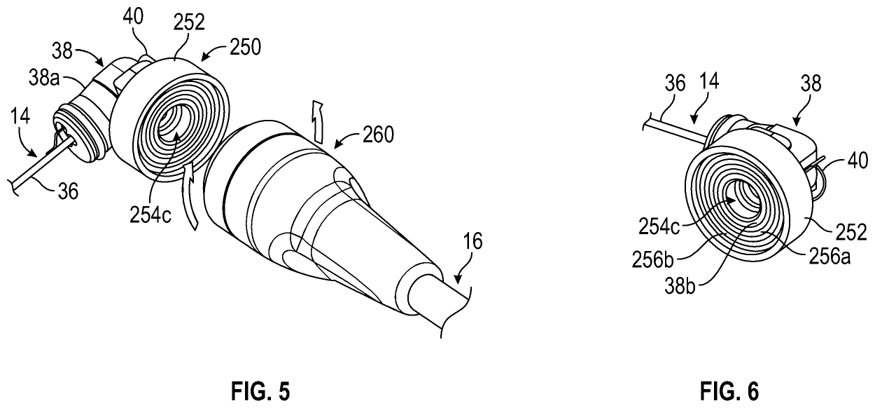

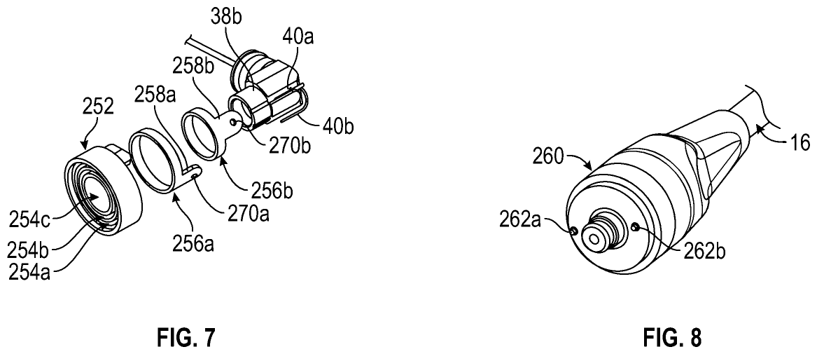

FIGS. 5-8 illustrate an alternate embodiment of first and second mating parts 250, 260 for detachably coupling the transmission line 16 and antenna assembly 14 to one another. In embodiments, the first and second mating parts 250, 260 may be incorporated into any of the embodiments of the devices described herein. The first and second mating parts 250, 260 are similar to the first and second mating parts 50, 60 described with reference to FIGS. 2, 3A, and 3B, and will therefore only be described in the detail necessary to elucidate selected distinctions.

The first mating part 250 includes a circular housing 252 defining a series of concentric, circular channels 254a, 254b and a central recess 254c for receipt of the second end portion 38b of the transition block 38. The housing 252 may be fixed about the second end portion 38b of the transition block 38. The first mating part 250 further includes a pair of conductive ring member 256a, 256b fixedly disposed in a corresponding circular channel 254a, 254b of the housing 252. Each of the ring members 256a, 256b has a finger 258a, 258b protruding therefrom. The finger 258a, 258b of each of the ring members 256a, 256b defines an opening 270a, 270b having received therein an end of a respective wire 40a, 40b of the temperature sensor 40 of the antenna assembly 14. It is contemplated that the wires 40a, 40b of the temperature sensor 40 are fixed and electrically connected to the fingers 258a, 258b of the ring members 256a, 256b.

The second mating part 260 has a pair of diametrically-opposed, conductive nodes 262a, 262b electrically coupled to the individual wires (not explicitly shown) of the thermocouple wire 44 (FIG. 2) of the transmission line 16. A first node 262a of the second mating part 260 is configured to be received in the first circular channel 254a of the first mating part 250, and a second node 262b is configured to be received in the second circular channel 254b of the first mating part 250, whereby each of the individual wires of the thermocouple wire 44 of the transmission line 16 are electrically coupled to the individual wires 40a, 40b of the temperature sensor 40 of the antenna assembly 14.

Upon coupling the first and second mating parts 250, 260 to one another, the transmission line 16 is rotatable about its longitudinal axis and relative to the antenna assembly 14 while remaining coupled to the antenna assembly 14. During rotation of the transmission line 16, the nodes 262a, 262b of the second mating part 260 travel around the respective circular channels 254a, 254b of the first mating part 250. The nodes 262a,262b and the ring members 256a, 256b may be fabricated from magnetic material to assist in maintaining the first and second mating parts 250, 260 coupled to one another via magnetic attraction.

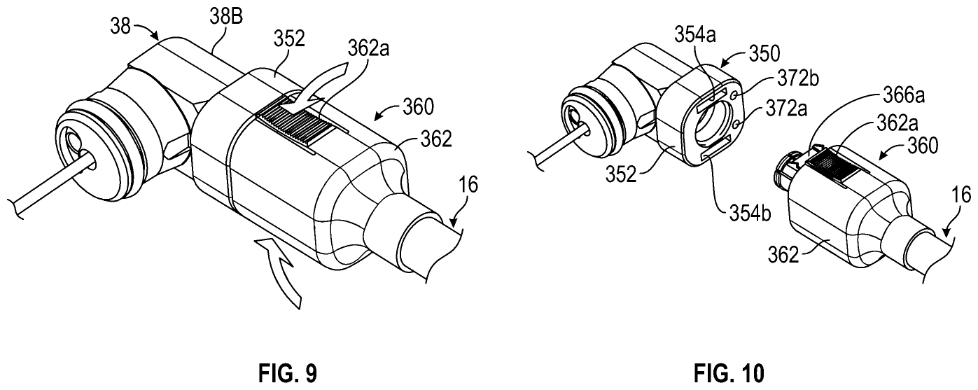

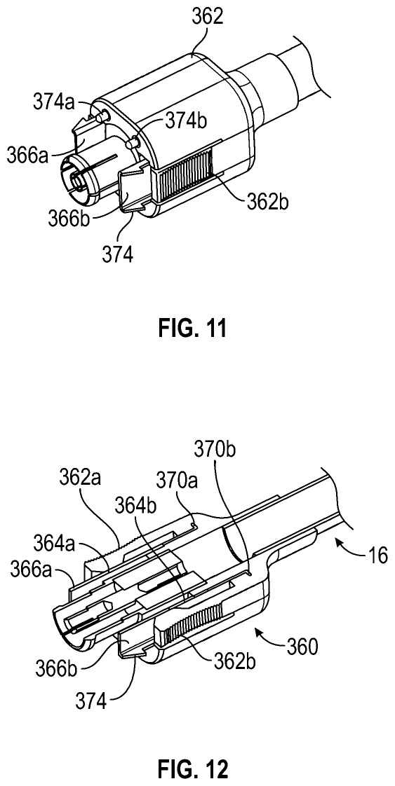

FIGS. 9-12 illustrate another alternate embodiment of first and second mating parts 350, 360 for detachably coupling the transmission line 16 and antenna assembly 14 to one another. In embodiments, the first and second mating parts 350, 360 may be incorporated into any of the embodiments of the devices described herein. The first and second mating parts 350, 360 are similar to the first and second mating parts 50, 60 described with reference to FIGS. 2A-2C, and will therefore only be described in the detail necessary to elucidate selected distinctions.

The first mating part 350 has a main body 352 fixed to the second end portion 38b of the transition block 38. The main body 352 defines a pair of diametrically opposed slits 354a, 354b disposed on opposite sides of the second end portion 38b of the transition block 38.

The second mating part 360 includes an outer housing 362 defining a pair of flexible tabs 362a,362b on opposite sides of the outer housing 362. The second mating part 360 includes a pair of flexible arms 364a, 364b disposed within the outer housing 362, such that the tabs 362a, 362b overlap the pair of arms 364a, 364b. The arms 364a, 364b are movable from a first state, in which free distal ends 366a, 366b of the arms 364a, 364b are out of alignment with the slits 354a, 354b of the first mating part 350 when the first and second mating parts 350, 360 are positioned for coupling, and a second state, in which the free distal ends 366a, 366b are spaced apart from one another the same distance the slits 354a, 354b are spaced apart from one another. Proximal ends 370a, 370b of the arms 364a, 364b resiliently bias the free distal ends 366a, 366b thereof toward the first state. The free distal ends 366a, 366b of the arms 364a, 364b each have a hook-shaped member 374, such as, for example, a barb, latch, protrusion, and/or tooth for facilitating fixation of the free distal ends 366a, 366b in the slits 354a, 354b.

To couple the first and second mating parts 350, 360 to one another, the first and second mating parts 350, 360 are positioned such that the free distal ends 366a, 366b of the arms 364a, 364b of the second mating part 360 are substantially aligned with the slits 354a, 354b of the first mating part 350 and the nodes 374a, 374b of the second mating part 360 are aligned with the holes 372a,372b of the first mating part 350. The tabs 362a,362b of the outer housing 362 are flexed inwardly, thereby flexing the arms 364a, 364b inwardly until the free distal ends 366a, 366b of the arms 364a, 364b are aligned with the slits 354a, 354b of the first mating part 350. The free ends 366a, 366b of the arms 364a, 364b are translated into the slits 354a, 354b, whereupon the free distal ends 366a, 366b pass over a corresponding lip (not shown) protruding into the slits 354a, 354b. At this point, the clinician may release the tabs 362a,362b of the second mating part 360, allowing the tabs 362a,362b and, in turn, the arms 364a, 364b, to flex outwardly back to their first state in which the free distal ends 366a, 366b catch on the lip to resist withdrawal of the transmission line 16 from the antenna assembly 14. To decouple the transmission line 16 from the antenna assembly 14, the tabs 362a,362b of the second mating part 360 are flexed inwardly to disengage the free distal ends 366a, 366b of the arms 364a, 364b from the lip, and the transmission line 16 is translated away from the transition block 38.

The above-described energy-delivery devices are capable of directing energy into tissue, and may be suitable for use in a variety of procedures and operations. The above-described energy-delivery devices may be suitable for utilization with hand-assisted, endoscopic and laparoscopic surgical procedures. The above-described energy-delivery devices may also be suitable for utilization in open surgical applications.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques).

What is claimed is:

1. An energy-delivery device for delivering energy to tissue, comprising:
a handle body;
an antenna assembly coupled to the handle body and extending distally therefrom;
a transmission line having a first end portion configured to be coupled to the handle body and a second end portion configured to be coupled to an energy source;
a first mating part coupled to the handle body or the first end portion of the transmission line; and
a second mating part coupled to the other of the handle body or the first end portion of the transmission line, wherein the second mating part is configured to be detachably coupled to the first mating part to detachably couple the transmission line to the antenna assembly.

2. The energy-delivery device according to claim 1, wherein the first mating part defines a recess and the second mating part has a projection configured for receipt in the recess to couple a coaxial cable of the transmission line to a feedline of the antenna assembly.

3. The energy-deliver device according to claim 2, wherein the first mating part defines at least one aperture and the second mating part has at least one node configured to be received within the at least one aperture to couple a thermocouple wire of the transmission line to a temperature sensor of the antenna assembly.

4. The energy-deliver device according to claim 2, wherein the projection includes a plurality of tabs arranged in a circular array, the plurality of tabs configured to flex radially inward upon receipt within the recess.

5. The energy-delivery device according to claim 4, wherein the first mating part includes a rod centrally disposed within the recess, and the second mating part includes a tubular member disposed within the plurality of tabs, the tubular member configured to receive the rod.

6. The energy deliver device according to claim 2, wherein the first mating part defines a pair of depressions disposed on opposite sides of the recess, and the second mating part has a pair of surface features disposed on opposite sides of the projection, the pair of depressions configured to receive the corresponding pair of surface features.

7. The energy-delivery device according to claim 1, wherein the first mating part is disposed at a proximal-facing end of the handle body and faces a proximal direction.

8. The energy-delivery device according to claim 1, wherein the first mating part is disposed at a proximal portion of the handle body and faces a direction that is perpendicular to a longitudinal axis defined by the antenna assembly.

9. The energy-delivery device according to claim 1, wherein the first and second mating parts are magnetically attracted.

10. The energy-delivery device according to claim 9, wherein the second mating part is rotatable relative to the first mating part while remaining coupled to the first mating part.

11. The energy-delivery device according to claim 10, wherein the first mating part defines a ring-shaped channel and the second mating part has at least one node configured for receipt within the ring-shaped channel.

12. The energy-deliver device according to claim 1, wherein the first mating part defines a pair of slits and the second mating part has a pair of flexible arms configured for receipt within the corresponding pair of slits.

13. A microwave-energy delivery device, comprising:
a handle body;
an antenna assembly coupled to the handle body and extending distally therefrom;
a transmission line having a first end portion configured to be coupled to the handle body and a second end portion configured to be coupled to an energy source;
a first mating part coupled to the handle body and defining a recess; and
a second mating part coupled to the first end portion of the transmission line and having a projection configured for receipt in the recess, wherein the first and second mating parts are magnetically attracted to one another and configured to detachably couple the transmission line to the antenna assembly.

14. The microwave-energy delivery device according to claim 13, wherein the projection includes a plurality of tabs arranged in a circular array, the plurality of tabs configured to flex radially inward upon receipt within the recess.

15. The microwave-energy delivery device according to claim 13, wherein the second mating part is rotatable relative to the first mating part while remaining coupled to the first mating part.

16. The microwave-energy delivery device according to claim 15, wherein the first mating part defines a ring-shaped channel and the second mating part has at least one node configured for receipt within the ring-shaped channel

17. The microwave-energy delivery device according to claim 13, wherein the first mating part defines a pair of slits and the second mating part has a pair of flexible arms configured for receipt within the corresponding pair of slits.

18. A method of using a microwave-energy delivery device, comprising:
inserting an antenna assembly of a microwave-energy delivery device into a body while the antenna assembly is detached from a transmission line of the microwave-energy delivery device; and
coupling a first mating part disposed at a first end portion of the transmission line to a second mating part disposed at a handle body of the microwave-energy delivery device after the antenna assembly is inserted into the body, thereby coupling a coaxial cable of the transmission line to a feedline of the antenna assembly and a thermocouple wire of the transmission line to a temperature sensor of the antenna assembly.

19. The method according to claim 18, wherein coupling the first and second mating parts includes magnetically coupling the first and second mating parts.

20. The method according to claim 19, wherein coupling the first and second mating parts includes inserting a projection of the first mating part into a recess defined by the second mating part.
